(19) United States
(12) Patent Application Publication
(10) Pub. No.: US 2026/0224153 A1
(43) Pub. Date: Aug. 6, 2026

(54) WAVELET CONSTRUCTION METHOD FOR BIOMEDICAL SIGNALS AND DEVICE FOR BIOMEDICAL SIGNALS

(71) Applicant: YASAR UNIVERSITESI, Izmir (TR)

(72) Inventors: Nalan OZKURT, Izmir (TR); Evrim SIMSEK, Izmir (TR); Cagla SARVAN CIBIL, Izmir (TR)

(73) Assignee: YASAR UNIVERSITESI, Izmir (TR)

(21) Appl. No.: 19/150,950

(22) PCT Filed: Jan. 23, 2024
(86) PCT No.: PCT/TR2024/050049

§ 371 Date: Jul. 25, 2025

(30) Foreign Application Priority Data
Jan. 26, 2023 (TR) 2023/000937

(51) Int. Cl. A61B 5/349; A61B 5/00

(52) U.S. Cl. CPC A61B 5/349; A61B 5/726

(57) ABSTRACT

A method of constructing a fundamental wavelet and wavelet family that specific to the relevant signal for the analysis of digitalized biomedical signals, and a biomedical device that provides anomaly detection with fundamental wavelet are provided. The method includes: creating a search space consist of more than one piecewise polynomial function, determining the most suitable piecewise polynomial by the genetic algorithm, ensuring orthogonality condition, representing the individual genotype as piecewise polynomials, generating new solutions/individuals by applying a crossover operation at the genotype level, obtaining discrete filter coefficients over the genotypes, representing the discrete filter coefficients as the phenotype of the genetic algorithm, evaluating the individuals/solutions according to the fitness value calculations assigned at the phenotype level, searching the most suitable discrete filters for the target signal, continuing this process until the termination criterion is met, constructing a new fundamental wavelet function as a result of the applied genetic algorithm.

### CROSS REFERENCE TO THE RELATED APPLICATIONS

This application is the national phase entry of International Application No. PCT/TR2024/050049, filed on Jan. 23, 2024, which is based upon and claims priority to Turkish Patent Application No. 2023/000937, filed on Jan. 26, 2023, the entire contents of which are incorporated herein by reference.

### TECHNICAL FIELD

The invention is about the reconstruction of signal-specific mother wavelet and wavelet family for the analysis of digital biomedical signals, and integration to the biomedical devices.

### BACKGROUND

Healthcare professionals use biomedical signals, especially electrocardiogram signals, to detect various disorders such as arrhythmias. This detection is usually based on the morphological characteristics of the signal. During these determinations, there are difficulties in the correct diagnosis of some waveforms due to noises such as muscle, power line, which are added to the signal and cannot be fully filtered.

There are various devices and software that are used by healthcare personnel which enable the classification of measured biomedical signals to overcome the analysis difficulties due to various noise sources. The aforementioned devices and software are expected to determine whether the distortions in the received signal are noise or a waveform deformation caused by arrhythmia, because this determination forms the basis of the diagnosis to be made by the healthcare professional. Based on this expectation, classification performance is very important. Wavelet transform techniques have been used to classify these biomedical signals, such as electrocardiogram signals, and to determine the discriminative features. In the known state of the technique, Symmlet and Daubechies wavelet families are frequently applied to ECG signals.

In one study, in order to distinguish the sinus rhythms of healthy patients from ECG of patients exhibiting arrhythmia, level 2 Discrete Wavelet Transform (DWT) coefficients were employed by using the Symmlet 5 (Sym5) wavelet. These extracted features were used in the classification algorithm [1].

In another study, a matching pursuit algorithm was applied to the signals for the discrimination of normal sinus rhythm, left bundle branch block, right bundle branch block, paced rhythm and premature ventricular contraction arrhythmias and suitable atoms of Symmlet 8 (Sym8) wavelet packet were selected by the algorithm [2].

In another study, using the Daubechies 4 (DB4) wavelet, the DWT coefficients were obtained from three different ventricular arrhythmias and given to the classification algorithm as a discriminative feature. [3] In another study, Daubechies 6 (DB6) wavelet was applied to ECG (Normal, LBBB, RBBB, Paced) signals due to its morphological similarity to the QRS complex. The combination of level 3, 4 and 5 detail coefficients of the DB6 wavelet were selected as discriminative features and tested in classification algorithms. [4]

In the known state of the art, adaptive wavelet studies for ECG signals include different methods in which classification algorithms are also adapted.

In one study, the Morlet wavelet function was used as the activation function of the artificial neural network due to its similarity to the ECG signal shape, and the P-Q-R-S-T waveforms were expressed with the Morlet mother wavelet. The discrimination of normal sinus rhythm and abnormal rhythms with this feature structure was proposed [5]. In another study, they selected 100 points that may be important from the raw ECG beat for 7 different types of ECG beat classification and applied them as input to Morlet wavelet function-based neurons in the artificial neural network [6].

When we look at these studies, it is tried to determine the appropriate wavelet function for ECG arrhythmia type among general and standard wavelet families that can be adapted to different applications. This leads to the limitations of being dependent on existing wavelets in the studies.

It is also observed that the information in the sub-frequency bands obtained for different arrhythmia types provides distinctive features with the application of different wavelet functions. In document CN114305438A, the genetic algorithm is described as a method that allows optimization problems that are difficult to solve with analytical techniques to be solved practically with techniques based on evolution. Therefore, it is used in many optimization applications. As mentioned in the report, genetic algorithm is used in this study to optimize a cellular automata model of ECG signal.

A system for arrhythmia detection is described in paper EP1724684A1. Daubechies principle and the use of Daubechies wavelets in noise reduction, and ECG classification applications of wavelet transform, are described.

When all these studies are analyzed, there is no clear consensus on the selection of the fundamental wavelet that increases the detection and classification performance. There is no accepted method to create a signal-specific adaptive wavelet.

As a result, all the problems mentioned above have made it necessary to make an innovation in the related field.

### SUMMARY

The main purpose of the invention is to construct a signal-specific fundamental wavelet to be used in the classification of biomedical signals in order to increase the performance.

A further objective of the invention is the construction of a fundamental wavelet specifically designed for electrocardiogramaignals.

Another aim of the invention is to construct a fundamental wavelet for arrhythmia detection. The aim of the invention is to increase the success in biomedical signal analysis, especially in arrhythmia detection.

The invention constructs a signal-specific wavelet for the signal to be classified.

First, a piece-wise polynomial that represents the waveform to be classified is constructed. Daubechies regularity condition is applied to the constructed polynomial and the nonorthogonal polynomials are ignored, then the filter coefficients are obtained. Aforementioned filter coefficients are evaluated with various fitness tests and the best coefficients are transferred to the genetic algorithm as parents. New fundamental wavelet is then reconstructed from these filter coefficients as the form of polynomials.

The reconstructed fundamental wavelet is employed as a feature extractor for the biomedical devices that classifies the biomedical signals.

Also, this application covers the integration of the wavelet construction algorithm

Furthermore, the present application also covers a device integration that runs the classification algorithm with extracted features which provided from mentioned constructed wavelet function. For instance, the presented method can be used in an electrocardiogram or wearable device, for instance a Holter device for increase the success.

### BRIEF DESCRIPTION OF THE DRAWINGS

To be better explanation of developed device with this invention, used figures and corresponding descriptions are listed in below.

FIG. 1 shows a flowchart of the process steps to obtain filter coefficients

FIG. 2 shows a flowchart of the genetic algorithm steps

FIG. 3 shows a graph of an ECG beat shape

### DETAILED DESCRIPTION OF THE EMBODIMENTS

Subject of invention is relevant to a method that is to construct a signal specific wavelet basis and wavelet family for analysis of biomedical signals and anomaly detection that can be used in biomedical devices.

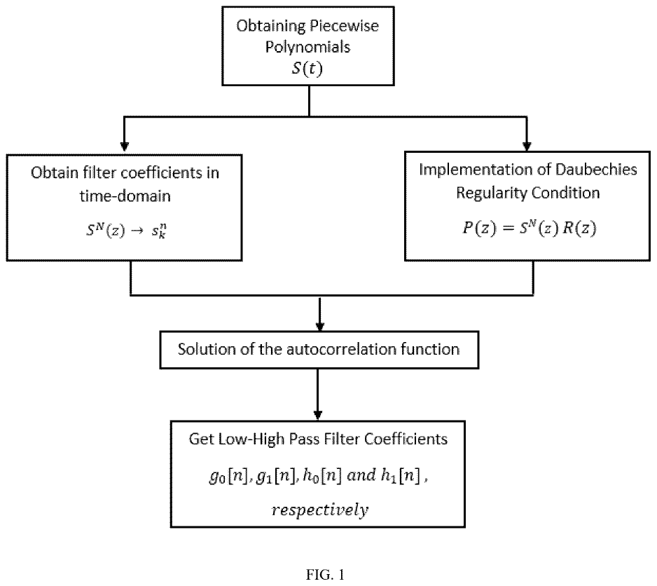

The mentioned digital biomedical signals can be any signal that used in medically defined fields. Preferably the mentioned signals are electrocardiogramaignals, mentioned wavelet basis constructed for detection of arrythmia in an algorithm that provides classification. Referring the FIG. 1: According to properties of the non-stationary signals, the problem is handling as an optimization problem that construct wavelet functions which satisfies the properties of the Nth order vanishing moments, linear phase, symmetry, regularity and smoothness. The piecewise polynomials, which provides these conditions and reflects the signal shapes, is produced from basis signals.

In this invention, a piecewise polynomial is obtained from an initial function according to the handled biomedical signal. In genetic algorithm, the generated piecewise polynomials at the initial of the algorithm, are defined as genotype.

If mentioned method is used for an electrocardiogramal, to reflect the signal's morphologic features, the biggest and smallest time interval and amplitude values of the P-Q-R-S-T wave peaks are used of a real ECG signal. Basis signal functions are defined as piecewise polynomials such as in equation (1). Then, by applying shifting and dilation to these functions, scaling and wavelet functions will be obtained to represent the P-Q-R-S-T waveforms of the given ECG signal type. The piecewise polynomials, that given in equation (1), are initialized randomly depending on the assigned time interval tᵢ and polynomial order Nₚ. Then, based on the applied evolutionary algorithm, it evolves in accordance with the morphological characteristics of the wave shape of the ECG beat.

The given piecewise polynomial S(t) will be assigned as initial function. This defined function is the genotype of the individual in the population.

$$S ( t ) = \{ \begin{matrix}{N}_{p} th order polynomial piece 1 & {t}_{0} \leq t < {t}_{1} \\ {N}_{p} th order polynomial piece 2 & {t}_{1} \leq t < {t}_{2} \\ 0 & otherwise\end{matrix} \qquad ( 1 )$$

The values given by the polynomials between the tᵢ values expressed in equation (1) (i.e. y-axis amplitude values) are assigned in accordance with the lower and upper amplitude limits of biomedical signals. It represents the degree of the given Nₚ piecewise polynomial.

The piecewise polynomials obtained as genotypes also define the initial scaling function. The initial scaling function is used as the basis function and the z-transform is applied to obtain the Nth order filter coefficients. The z-transform of the piecewise polynomial function is formulated as in equation (2). By applying the obtained Nth order filter coefficients in the Daubechies regularity principle, low and high pass filter coefficients to be applied in the Discrete Wavelet transform are created. These filter coefficients obtained from piecewise polynomials in the solution space are defined as phenotypes in the genetic algorithm.

In the given equation (2), PN-1 represents the value at which the polynomial equation ends on the time axis as an integer. The multipliers expressed as s[k] represent the filter coefficients.

$$ℤ \{ s [ n ] \} = S ( z ) = {\sum }_{k = 0}^{PN - 1} s [ k ] {z}^{- k} \qquad ( 2 )$$

In equation (2), S(z) represents the first order FIR filter that obtained from the Nₚth order piecewise polynomial. To obtain Nth order filter, equation (3) is applied.

$${S}^{N} ( z ) = {( {s}_{0} + {s}_{1} z )}^{N} \qquad ( 3 )$$

Daubechies' regularity condition is applied to produce filter coefficients that satisfy the regularity condition in the wavelet theory. The design procedure for Daubechies wavelets involves finding orthogonal low-pass filters that contain a many number of zeros. An autocorrelation equation equivalent to this situation is expressed as follows: A symmetric R(z) function is added as a multiplier to the z-transform of the Haar fundamental wavelet. In the mentioned invention, the Haar basis function is replaced with the Nth order filter obtained with the Piecewise Polynomial structure and the new autocorrelation process obtained is expressed by the following mathematical equation.

$$P ( z ) = {G}_{0} ( z ) {G}_{0} ( {z}^{- 1} ) = {S}^{N} ( z ) R ( z ) \qquad ( 4 )$$

Although the Daubechies regularity condition, which was explained in this section, was applied to construct of the Daubechies wavelet family, it is specifically adapted to piecewise polynomial structures in a different context in this mentioned invention.

Factorization of the autocorrelation function that given in Equation (4) produces R(z) coefficients and G₀(z) is found from the R(z) function. By taking the inverse z-transform of G₀(z), the low-pass filter coefficients g₀[n] that used to reconstruct the signal in wavelet theory, is obtained. The high-pass filter coefficients g₁[n] are found from the low-pass filter coefficients g₀[n] by the mathematical equation (5) that given in below.

$${g}_{1} [ n ] = {( - 1 )}^{n} {g}_{0} [ - n + 1 ] \qquad ( 5 )$$

Thus, the scaling and wavelet functions obtained by using shifting and dilation of the initial function, which is the genotype of the individual in the genetic algorithm, and the filter coefficients of these functions express the phenotype of the individual.

After the calculation of the g₀[n] coefficients, the corresponding piecewise polynomial representations, that is the initial scaling function and also the genotype of the individual, where the filter coefficients were obtained, are used.

$$∅ ( t ) = S ( t ) = \{ \begin{matrix}{a}_{11} {t}^{{N}_{p}} + {a}_{12} {t}^{{N}_{p} - 1} + … + {a}_{1 ( {N}_{p} + 1 )} & {t}_{1} \leq t < {t}_{2} \\ {a}_{21} {t}^{{N}_{p}} + {a}_{22} {t}^{{N}_{p} - 1} + … + {a}_{2 ( {N}_{p} + 1 )} & {t}_{2} \leq t < {t}_{3} \\ 0 & aski halde\end{matrix} \qquad ( 6 )$$

Nₚth order piecewise polynomial is taken as the initial scaling function. Nₚth order piecewise polynomial function is expressed in Equation (6). The values g₀[n] and g₁[n] are used as in Equations (7) and (8), respectively, to express the scaling and wavelet functions from the piecewise-polynomial function that generated as genotype, in the continuous time form, respectively:

$${∅}^{( i )} ( t ) = \sqrt{2} {\sum }_{k} {g}_{0} [ k ] {∅}^{( i - 1 )} ( 2 t - k ) \qquad ( 7 )$$

$${φ}^{( i )} ( t ) = \sqrt{2} {\sum }_{k} {g}_{1} [ k ] {∅}^{( i )} ( 2 t - k ) \qquad ( 8 )$$

High and low pass filter coefficients are obtained from the scaling function. The resulting scaling and wavelet functions and their filter coefficients express as the phenotype of the individual. The genotype of this phenotype is stored in the algorithm for each individual in terms of aij coefficients and the order Np value, with the representation given in equation (6).

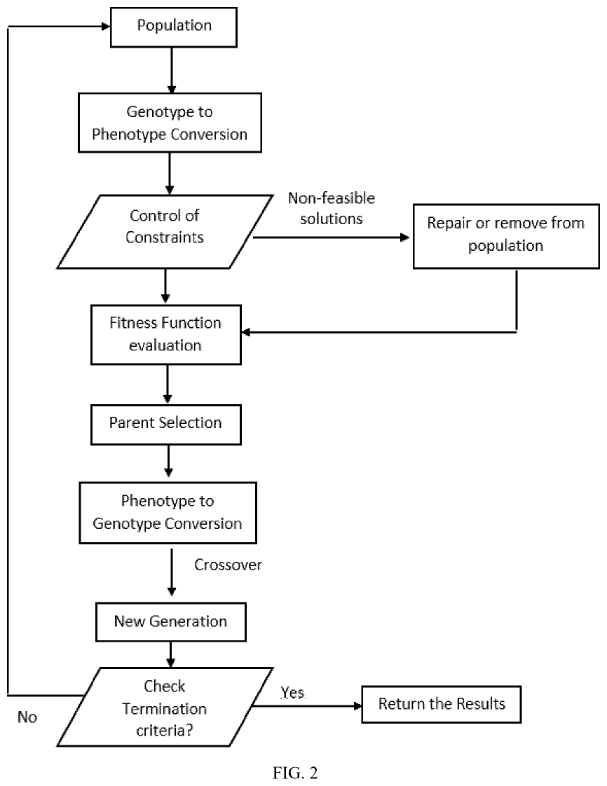

Referring to FIG. 2; A group (population) is created by determining the filter coefficients as phenotypes. The mentioned group was prepared to be used in a genetic algorithm. The group enter the orthogonality check before using in genetic algorithm.

Here, preferably the non-orthogonal element is entered to orthogonalization process. Preferably, the Gram-Schmidt orthogonalization procedure is used in this step.

In a preferred procedure, the elements that cannot be orthogonalized are removed from the population during the orthogonalization process.

Alternatively, non-orthogonal elements are directly removed from the group without performing any orthogonalization process.

The low-high pass filter coefficients represent an individual in the population. The number of produced individuals in the population is M, and these parameter assignments are made at the beginning of the algorithm.

At the beginning, it is checked once whether each individual in the population complies with the constraints. The constraint equations are given in Equations (9), (10) and (11), respectively. Individuals who do not comply are removed from the population or are repaired, that is, orthogonalized to comply with the constraints.

$$\begin{matrix}( iv ) & 〈 {g}_{i} [ n - 2 k ] , {g}_{j} [ n - 2 l ] >= δ [ i - j ] δ [ k - l ]\end{matrix} \qquad ( 9 )$$

$$\begin{matrix}( v ) & 〈 {h}_{i} [ n - 2 k ] , {h}_{j} [ n - 2 l ] 〉 = δ [ i - j ] δ [ k - l ]\end{matrix} \qquad ( 10 )$$

$$\begin{matrix}( vi ) & 〈 {h}_{i} [ n ] , {g}_{i} [ n - 2 k ] 〉 = 〈 {g}_{i} [ n ] , {h}_{i} [ n - 2 k ] 〉 = 0\end{matrix} \qquad ( 11 )$$

As the construction progresses in the algorithm, individuals in the new generation are subjected to constraint control again and control is provided by repairing or removing individuals that do not comply with this control. Individuals who pass the control are processed for the implementation of the next stage.

After orthogonalization control and regularity condition process, the fitness values of the coefficients are calculated, and according to the selection algorithm applied in the multi-objective genetic algorithm, coefficients are selected based on their fitness values as parents that will produce new individuals (solutions).

During the calculation of fitness values, more than one fitness value is calculated and applied to the multi-objective genetic algorithm.

One of these fitness values is extraction of the detail coefficients at different scaling levels which are obtained by applying constructed wavelet functions to the targeted ECG beat types in Discrete Wavelet Transform algorithm. Here, energy calculation is applied by using the level-based detail coefficients to determine how well the obtained values match the signal shape to be analyzed.

In another fitness value is the Wavelet Coherence value, which measures the similarity between the obtained wavelet function and the biomedical signal.

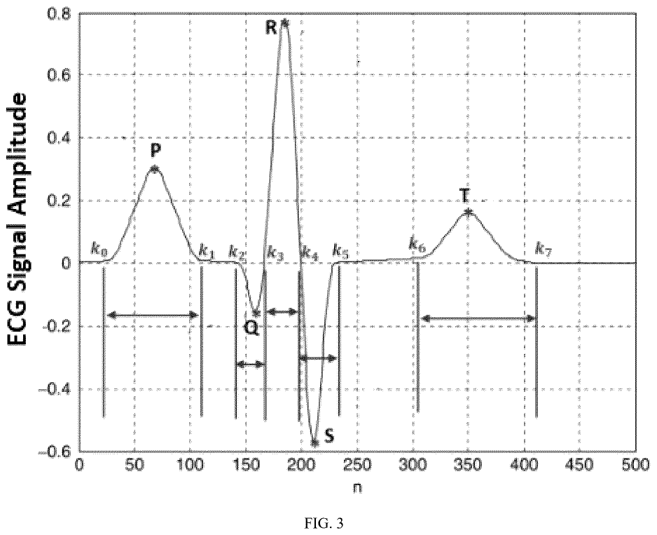

For another fitness value of the multi-objective genetic algorithm is the average values of the amplitude and time intervals of the P-Q-R-S-T waveforms obtained from real ECG signal measurements are used for morphological similarity measurement. Here, the difference between the morphological features of the wavelets obtained with the proposed algorithm and the average of the real signals is tried to be reduced as much as possible. Referring to FIG. 3; The mentioned graph shows an electrocardiogramal. kᵢ values represent the time intervals of the waveforms and points of P-Q-R-S-T represents the amplitude values.

As the final fitness value, the statistical properties of the detail coefficients are assigned as features and given to the classifier to calculate the percentage rates of accuracy, specificity and sensitivity.

In the proposed invention, due to the nature of the multi-objective genetic algorithm, solutions are produced for all mentioned fitness function calculation methods. All fitness values are calculated, including at least two fitness values. The user chooses among the solutions whichever suitability value is important according to his/her application. For example, if classification accuracy and morphological similarity are more important in practice, results with high values of these finesses may be preferred.

At least two or preferably all of the fitness value calculation methods mentioned here can be used together.

Thus, the wavelet functions that will reflect the ECG signal beat types is produced through a multi-objective genetic algorithm in line with the above fitness functions in terms of both time and frequency and classification algorithms.

In a ranking of the fitness values mentioned, the coefficients that give at least the two best results are selected and defined as parents in a genetic algorithm. By exchanging genes between the mentioned parents, a new piecewise function is produced as a fundamental wavelet. The individuals produced enter the crossover process for new solutions. The crossover process is carried out at the genotype level. Genotypes are expressed as piecewise polynomials. For this reason, new different two-piece polynomials are obtained by taking the parts of the piecewise polynomials at certain time intervals as genes, and the new piecewise polynomials mentioned here express the fundamental signal specific wavelets.

The elements of this resulting piecewise functions are defined as individuals of new generation (new population).

The creation of new generations of individuals continues until a termination criterion is met and then ends. The termination criterion applied in the algorithm can be assigned as the number of generations or reaching the targeted value in the fitness functions.

### REFERENCES

[1] Wiggins, M., Zhao, L., Vachtsevanos, G., Litt, B. 2003. “Non-invasive, cardiac risk stratification using wavelet coefficients”, WSEAS Transactions on Computers, 2, 720-723.
[2] Christov, I., Gómez-Herrero, G., Krasteva, V., Jekova, I., Gotchev, A., Egiazarian, K. 2006. “Comparative study of morphological and time-frequency ECG descriptors for heartbeat classification”, Medical engineering & physics, 28 (9), 876-887.
[3] Arumugam, S. S., Gurusamy, G., Gopalasamy, S. 2009. “Wavelet based detection of ventricular arrhythmias with neural network classifier” Journal of Biomedical Science and Engineering, 2 (06), 439.
[4] Sahoo S., Kanungo B., Behera S., Sabut S. 2017. “Multiresolution wavelet transform based feature extraction and ECG classification to detect cardiac abnormalities”, Journal
[5] Kadambe, S., Srinivasan, P. 2006. “Adaptive wavelets for signal classification and compression”, AEU-International Journal of Electronics and Communications, 60 (1), 45-55.
[6] Lin, C. H., Du, Y. C., Chen, T. 2008. “Adaptive wavelet network for multiple cardiac arrhythmias recognition”, Expert Systems with Applications, 34 (4), 2601-2611.

What is claimed is:

1. A signal-specific, temporally non-stationary fundamental wavelet construction method for an analysis of time-varying and non-stationary digitalized biomedical signals, comprising:
creating a piecewise polynomial function representing a waveform of a target biomedical signal beat,
obtaining a solution space comprising-containing a low pass filter coefficient and a high pass filter coefficient with Daubechies regularity principle,
calculating fitness values of the low pass filter coefficient and the high pass filter coefficient and selecting at least two parents from a population according to the fitness values, wherein the at least two parents give best results according to their fitness values, and
changing polynomial pieces of the at least two selected parents from each other as genes and applying a crossover process to generate a piecewise function as individual genotypes.

2. The signal-specific, temporally non-stationary fundamental wavelet construction method according to claim 1, further comprising: removing solutions of corresponding low and high pass filter coefficients without an orthogonality property, and calculating fitness values of remaining filter coefficients.

3. The signal-specific, temporally non-stationary fundamental wavelet construction method according to claim 2, wherein an orthogonalization process is applied to elements of the solution space.

4. The signal-specific, temporally non-stationary fundamental wavelet construction method according to claim 3, wherein the orthogonalization process is provided depending on Gram-Schmidt orthogonalization procedure.

5. The signal-specific, temporally non-stationary fundamental wavelet construction method according to claim 2, wherein elements that could not orthogonalized are removed from the solution space.

6. The signal-specific, temporally non-stationary fundamental wavelet construction method according to claim 1, wherein the low pass filter coefficient and the high pass filter coefficient are applied to non-stationary signals in a wavelet transform before a calculation of the fitness values.

7. The signal-specific, temporally non-stationary fundamental wavelet construction method according to claim 6, wherein one of the fitness values is detected according to Wavelet Coherence method, wherein the Wavelet Coherence method measures similarities between a wavelet function and the non-stationary signals.

8. The signal-specific, temporally non-stationary fundamental wavelet construction method according to claim 1, wherein one of the fitness values is detected according to a morphological similarity measurement.

9. The signal-specific, temporally non-stationary fundamental wavelet construction method according to claim 1, wherein one of the fitness values is detected according to an energy calculation of level-based detail coefficients.

10. The signal-specific, temporally non-stationary fundamental wavelet construction method according to claim 1, wherein one of the fitness values calculates features obtained from level-based detail coefficients, and the features are given to a classifier to determine results by comparing an accuracy percentage rate, a specificity percentage rate, and a sensitivity percentage rate.

11. The signal-specific, temporally non-stationary fundamental wavelet construction method according to claim 1, wherein in a multi-objective genetic algorithm model, the at least two parents are selected from the population as a result of at least two fitness value calculation methods.

12. The signal-specific, temporally non-stationary fundamental wavelet construction method according to claim 11, wherein more than two fitness value calculation methods are used.

13. The signal-specific, temporally non-stationary fundamental wavelet construction method according to claim 1, wherein the crossover process continues to produce two-piece polynomials until a termination criterion is met.

14. The signal-specific, temporally non-stationary fundamental wavelet construction method according to claim 13, wherein the crossover process continues to obtain different two-piece polynomials until the termination criterion of completing a number of generations or reaching a targeted value in fitness functions is met.

15. The signal-specific, temporally non-stationary fundamental wavelet construction method according to claim 1, wherein the piecewise polynomial function representing the waveform of the target biomedical signal beat is based on B-spline.

16. A method for an anomaly detection with a fundamental wavelet constructed by the signal-specific, temporally non-stationary fundamental wavelet construction method according to claim 1, comprising performing over a signal received by a biomedical reader.

17. The method according to claim 16, wherein a basic wavelet is applied as a main wavelet and a wavelet family for obtained wavelet coefficients of the signal to a wavelet analysis of the signal and the anomaly detection, wherein the obtained wavelet coefficients are configured for a feature extraction in a classification.

18. The method according to claim 16, wherein the biomedical reader is an electrocardiogram.

19. The method according to claim 16, wherein the biomedical reader is a wearable device.

20. The method according to claim 16, wherein the biomedical reader is a holter device.

21. (canceled)

22. (canceled)
